(19) United States
(12) Patent Application Publication
(10) Pub. No.: US 2011/0000489 A1
(43) Pub. Date: Jan. 6, 2011

(54) CONTROL UNIT, METHOD AND COMPUTER-READABLE MEDIUM FOR OPERATING A VENTILATOR

(75) Inventors: Joakim Laksov, Danderyd (SE); Johan Lagerborg, Ronninge (SE)

Correspondence Address: SCHIFF HARDIN, LLP;PATENT DEPARTMENT, 233 S. Wacker Drive-Suite 6600, CHICAGO, IL, 60606-6473, US

(73) Assignee: MAQUET CRITICAL CARE AB, Solma (SE)

(21) Appl. No.: 12/809,691

(22) PCT Filed: Dec. 20, 2007
(86) PCT No.: PCT/SE2007/051048

§ 371 Date: Sep. 20, 2010

(51) Int. Cl. A61M 16/00

(52) U.S. Cl. 128/204.23

(57) ABSTRACT

To set trigger conditions correctly in pneumatic mode, a ventilator is controlled to obtain a measurement value of a bioelectric signal representative of the patient's breathing function, determine, based on the bioelectric signal, at least one point in time at which the patient starts inhalation, obtain a measurement value to be used for triggering an inspiration phase in the ventilator during the at least one point in time, determine a trigger condition for the inspiration phase on the basis of the measurement value, and use the trigger condition for initiating inspiration when ventilating the patient in support mode.

### BACKGROUND OF THE INVENTION

1. Field of the Invention

The present invention relates to a ventilator for use in support mode and a method for ventilating a patient in support mode.

2. Description of the Prior Art

A ventilator for providing breathing support to a patient can work in different modes, depending, i.e. on the patient's condition. If the patient is showing some breathing activity a support mode is often suitable, in which the ventilator provides extra breathing support in phase with the patient's own breathing activity. In this case the patient's own breathing activity must be monitored in an appropriate way in order to synchronize the breathing support provided by the ventilator with the patient's own breathing so that an inspiration phase is started by the ventilator when the patient starts inhaling. Typically a pneumatic trigger condition based on pressure and/or flow in the ventilator is set.

In some cases it is difficult to synchronize the ventilation correctly with the patient's breathing efforts. For example, if there is a leakage, it will be difficult to set a suitable pneumatic trigger level. Finding a suitable level may require a lot of trial and error. In particular, when ventilating children leakages generally occur, since in that case a cuff is typically not used around the tube.

Imperfect synchronization between the ventilator's and the patient's breathing cycles can lead to increased work for the patient. If the trigger is too insensitive an entire breath may be skipped. If the trigger is too sensitive an inspiration may be triggered in the ventilator when the patient is not ready to inhale.

### SUMMARY OF THE INVENTION

It is an object of the invention to improve the trigger conditions when ventilating a patient in support mode

This object is achieved according to the present invention by a computer readable medium having stored thereon computer readable code for controlling a ventilator providing breathing support in a support mode to a patient code including computer readable instructions which, when run in a control unit controlling a ventilator will cause the control unit to
obtain a measurement value of a bioelectric signal representative of the patient's own breathing function,
determine, based on the bioelectric signal, at least one point in time in which the patient starts inhalation,
obtain a measurement value to be used for triggering an inspiration phase in the ventilator during said at least one point in time,
determine a trigger condition for the inspiration phase on the basis of the measurement value, and
use the trigger condition to for initiating inspiration when ventilating the patient in support mode.

The object is also achieved by a method of controlling a ventilator providing breathing support in a support mode to a patient, characterized by the steps of
measuring a bioelectric signal representative of the patient's own breathing function;
determining, based on the bioelectric signal, at least one point in time in which the patient starts inhalation,
measuring the pressure in the ventilator during said at least one point in time,
determining a trigger condition for the inspiration phase on the basis of the measured pressure.

According to the invention, the Edi signal is used to determine the point in time when the patient starts inhaling, and the triggering conditions for an inspiration phase in the ventilator may be adjusted based on measurements of pressure and/or flow performed in the ventilator at this point in time. The invention therefore facilitates ventilation in pneumatic mode that is adapted to the patient's own breathing cycle.

As will be understood by those skilled in the art, the method is performed by a computer program, preferably located in the control unit of the ventilator for controlling the ventilator. Hence, the invention also relates to a control unit for a ventilator comprising a computer program product as defined above and a ventilator having such a control unit.

Preferably, the control unit will be caused to adjust the trigger condition by an amount determined on the basis of the measurement value.

In one embodiment the encoded instructions the control unit to determine the trigger condition on the basis of several measurement values, each obtained at a point in time when the patient starts inhalation, in different breaths. This will provide a more accurate value for the trigger condition.

In a preferred embodiment, the computer readable instructions which, when run in a control unit controlling a ventilator will cause the control unit, after determining the start of inhalation and before measuring the pressure, to determine whether an inspiration phase in the ventilator was triggered before the start of inhalation and, if so, to slightly delay the ventilator's inspiration phase until the patient's own breathing attempt can be detected.

In one embodiment the code instructions cause the control unit to adjust the trigger condition incrementally in such a way as to reduce the time difference between start of the ventilator's inspiration phase and the start of the patient's inspiration. The trigger condition may be adjusted, for example, in fixed increments or in increments determined on the basis of the difference between the measurement value and the trigger condition.

### BRIEF DESCRIPTION OF THE DRAWINGS

FIG. 1 illustrates a ventilator providing breathing support to a patient.

FIG. 2 illustrates a situation where the trigger condition is too sensitive.

FIG. 3 illustrates a situation where the trigger condition is too insensitive.

FIG. 4 is a flow chart of an embodiment of the inventive method.

FIG. 5 is a more detailed flow chart of one of the steps of FIG. 4.

### DESCRIPTION OF THE PREFERRED EMBODIMENTS

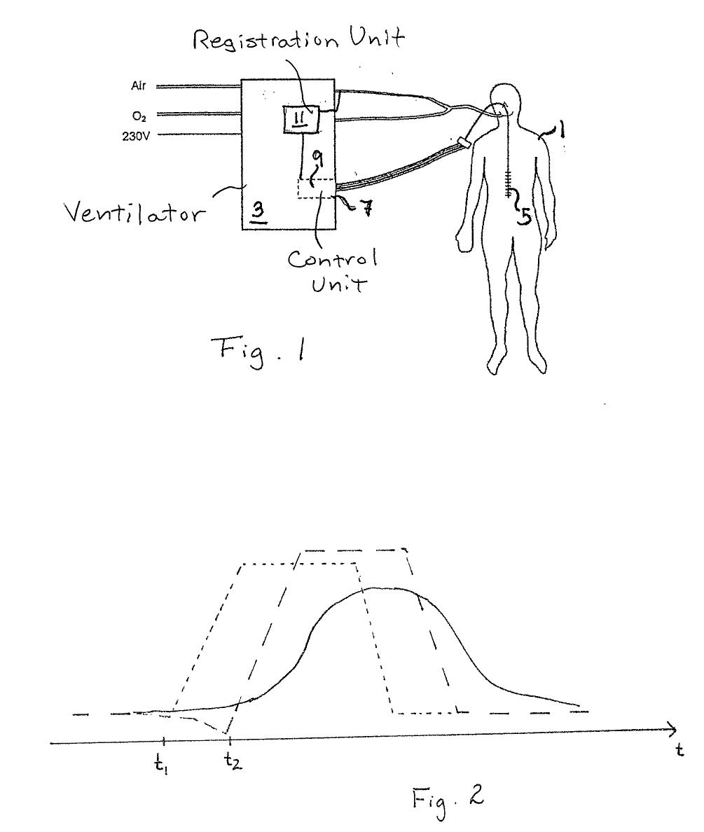

FIG. 1 is a schematic overview of a patient 1 connected to a ventilator 3. The ventilator is arranged to work in support mode but can also be arranged to work in a controlled mode. To capture the Edi signal, the patient 1 has an oesophageal catheter 5 inserted in order to record a myoelectric signal from the diaphragm. This myoelectric signal (EMG signal) is fed to a control input 7 of the ventilator 3 and processed in a control unit 9 in the ventilator to produce the overall signal, called an Edi signal. According to the invention, a bioelectric signal related to breathing, such as the Edi signal is used to adjust the pneumatic triggering criteria of the ventilator.

Typically the ventilator has registration unit 11 for monitoring the pressure and/or flow of breathing gas in the breathing circuit 1. The control unit 9 has a processor and at least one computer program that is executed to compare the patient's own breathing activity to the breathing support provided by the ventilator and to adjust, if necessary, the triggering of the inspiration to synchronize it better with the patient's own breathing. This will be discussed in more detail below.

In order to assist the patient's breathing in such a way that the patient's own attempts to inhale air are supported by an additional flow of breathing gas from the ventilator, the flow and/or pressure in the ventilator is measured. The pressure and/or flow sensors in the ventilator are used together with the trigger settings in order to detect the patient's attempt to inhale and initiate the inspiration phase. This is referred to as pneumatic triggering. Alternatively, an increased flow in the direction towards the patient is measured. Ideally the start of this inspiration phase should be perfectly synchronized with the start of the patient's own inhalation. To achieve this, the threshold value for the flow and/or pressure must be set correctly so that the flow and/or pressure measured in the ventilator will pass the threshold at exactly the same time as the patient starts inhaling. This is not always the case, as will be discussed in the following.

FIG. 2 illustrates a situation in which the pneumatic trigger function is based on pressure measurements and is too sensitive, causing the inspiration to be triggered too early in the breathing cycle compared to the patient's own breathing. Three curves are shown varying along a time axis denoted t. The solid curve represents the Edi signal recorded in the patient, that is, it reflects the patient's own breathing activity. The positive flank represents an inhalation by the patient. The dashed curve is an ideal ventilator cycle, starting inspiration (positive flank) when the patient starts inhaling. The dotted curve is an example of the breathing support that will result if the trigger condition is too sensitive. In this case, the triggering should be made to start later to be in phase with the patient's own breathing.

Too early triggering, as illustrated in FIG. 2, may be caused, for example, by a leakage in the breathing circuit or if there is water in the tubes. It may also be due to oscillations caused by variations in the patient's thorax, caused by heart activity. In the case shown in FIG. 2, the pressure will drop below the trigger condition at a first point in time t1, which occurs before the patient actually starts inhaling, at a second point in time t2. Hence, the ventilator will start inspiration support before the patient is ready to inhale. In this case, therefore, the pneumatic triggering should be delayed to be in phase with the patient's own breathing.

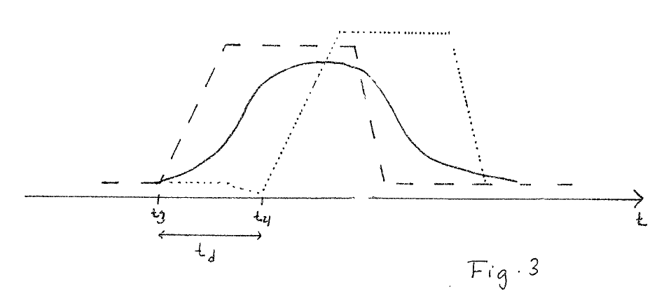

FIG. 3, like FIG. 2, illustrates a situation in which the pneumatic trigger function is based on pressure measurements. In FIG. 3, the trigger function is not sensitive enough, causing the inspiration to be triggered too late in the breathing cycle compared to the patient's own breathing. Three curves are shown varying along a time axis denoted t. As in FIG. 2, the solid curve represents the Edi signal recorded in the patient, that is, it reflects the patient's own breathing activity. The positive flank represents an inhalation by the patient. The dashed curve is an ideal ventilator cycle, starting inspiration (positive flank) when the patient starts inhaling. In FIG. 3, the dotted curve is an example of a delayed triggering that will result if the trigger condition is too insensitive. In this case, when the patient starts inhaling, at a point in time t3, the pressure will drop but not enough to trigger an inspiration phase in the ventilator at once. Only at a second point in time t4 will the ventilator start its inspiration phase. Hence, there will be a time delay td between the point in time t3 when the patient starts to inhale and the point in time t4 when the ventilator starts an inspiration.

In both the cases illustrated in FIGS. 2 and 3, the correct triggering point in time, that is the point in time when the patient starts to inhale, can be determined by means of an Edi signal recorded on the patient. By monitoring the Edi signal, the point in time when the patient starts to inhale can be determined, as the start of the positive flank of the Edi signal shown in FIGS. 2 and 3.

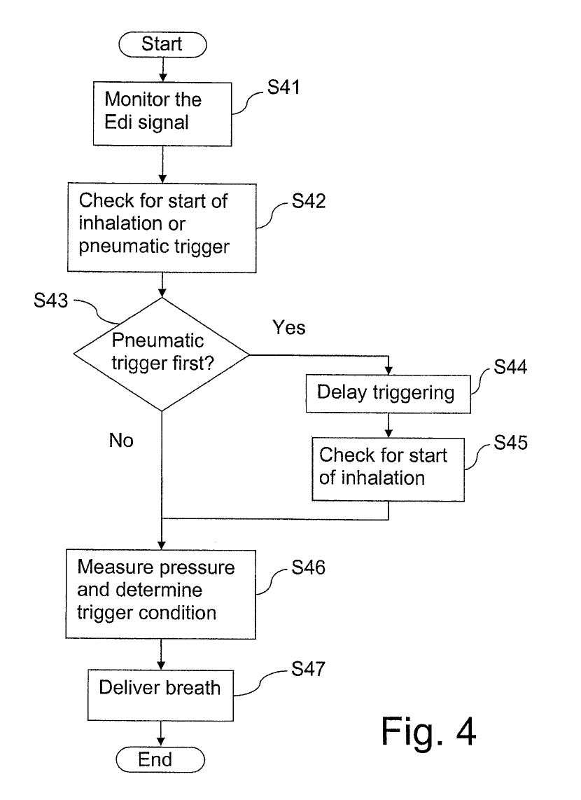

A first preferred embodiment of the inventive method is shown in FIG. 4. In this embodiment, as well as FIGS. 2 and 3 above, the triggering is based on pressure measurements. Those skilled in the art can easily modify this to triggering on flow criteria instead, or on a combination of flow and pressure criteria, if the ventilator supports this.

To initiate the method, in step S41, the Edi signal is monitored during at least one breath in the patient. In step S42, either the point in time when the Edi signal indicates patient inhalation during this breath, is determined, that is, the point in time in which the Edi signal raised above a certain predetermined value, or the point in time when the pneumatic trigger condition is reached, whichever occurs first. In step S43, preferably, it is determined if the ventilator is triggered before the start of inhalation. If yes, the triggering has to be delayed, in step S44. The point in time when the Edi signal indicates patient inhalation is then determined in step S45. After step S45, or after step S43 if the triggering was not too early, the pressure in the ventilator at the starting time of inhalation is measured in step S46. This pressure, that is, the pressure at the actual start of inspiration by the patient, is used in step S46 as an indicator of what the pneumatic trigger condition should be. Finally, in step S47 the new trigger condition is set to be used in the following breaths, or presented to the operator as a proposed new setting.

The method may be performed during one breath only, or may be performed during several breaths to obtain an average measured value. Such an average value will probably provide a more correct value of the pressure in the ventilator at the onset of the patient's own inspiration than a measured value obtained during only one breath. In both cases, the method may be performed again at certain time intervals to ensure correct timing of the breathing support. Alternatively, the procedure may be performed again if the difference between the start of the breathing cycle of the ventilator and that of the patient becomes too big.

The adjustment procedure may be initiated by an operator. Instead of automatic adjustment, the operator can also use the result to adjust the trigger condition manually, thereby adjusting the timing of the breathing support cycle.

In step S44 the triggering of the ventilator should be delayed so as to enable correct measurement of the pressure and/or flow at the point in time when the patient starts to inhale. Therefore, the triggering should not be performed until after the patient's inhalation has started. However, a maximum delay should be set, to ensure that the breathing support delay will not be harmful to the patient.

The correction of the trigger condition, based on the value determined in step S47, may be carried out in different ways. The trigger condition, which, in the case of pressure triggering, will be a pressure value, which may be set as a function of the pressure measured in step S46.

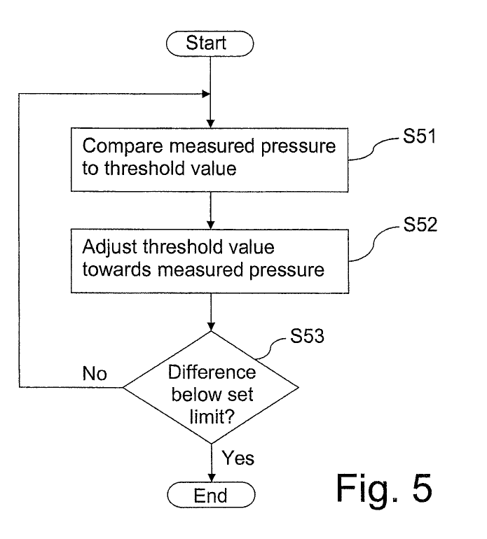

It may be favourable to adjust the trigger condition in several steps. In this case, step S47 will comprise the following substeps, illustrated in FIG. 5:

In step S51 comparing the pressure measured in step S43 to the actual trigger condition currently applied in the ventilator .

In step S52 adjusting the trigger condition in the direction of the measured pressure value. If the measured pressure value is lower than the pressure value that will trigger the inspiration phase, the threshold value should be lowered. If the measured pressure value is higher than the threshold pressure that will trigger the inspiration phase, the threshold value should be raised. The change in the threshold value may, however, be carried out stepwise, so that the trigger conditions will be refined gradually. The steps could be carried out, for example, in fixed increments, for example, 0.1 cmH2O at a time, or as a fraction of the difference, for example 10% of the determined difference each time. The procedure may be iterated a predetermined number of times, or until the difference between the trigger conditions and the measured pressure is within an acceptable interval. This is indicated by decision step S53 in FIG. 5, which terminates the procedure if the difference is below a set limit and returns to step S51 if the difference is still too large. Instead of determining the difference between actual pressure and threshold value, in the decision step S53 the difference in time between the start of the patient's own inhalation and the start of the inspiration phase of the ventilator could be evaluated, that is, the difference between the first and second points in time t1 and t2, or the difference between the third and fourth points in time t3 and t4, as the case may be. In this case, if the time difference is longer than a predetermined time, for example 100 ms, the procedure of FIG. 5 should be reiterated. The predetermined time could be of the order of magnitude of 100 ms. It may be determined as a fix value, or based on duration of the patient's own breathing cycle, or inspiration phase.

Preferably, a pressure and/or flow interval is defined in which the trigger condition can be set, to avoid setting the trigger condition to a value that may be harmful to the patient.

Also, in step S44 a maximum delay should be set for the pneumatic triggering to avoid losing an entire breath. This maximum delay could be, for example 300 ms. It could also be based on a measured duration of the patient's breathing cycle or inspiration phase. If no Edi triggering has occurred after the maximum delay, then the triggering value could be set to the value measured in the ventilator at the maximum delay and this could be used as an initial value. If no breathing activity can be detected from the Edi signal, a breath should still be delivered to the patient within a suitable time.

A minimum pressure should be set, which will always trigger the ventilator, even when the maximum delay has not been exceeded. This should correspond to the least sensitive pressure that is allowed.

In order to evaluate the result of the adjustment, the time difference between the pneumatic control of the ventilator and the Edi signal may be determined continuously or at certain time intervals. In this way the changes in the time difference over time can be monitored and appropriate action can be taken when needed.

The Edi signal is prone to disturbances, for example, from stronger bioelectric signals in the patient's body. To avoid using an erroneous Edi signal as a basis for the trigger conditions, an automatic adjustment should not be allowed if the time difference between inspiration phase triggered by the ventilator and the patient's own inhalation is too great. Alternatively a quality indicator for the Edi signal could be used, to ensure that the Edi signal actually reflects the patient's breathing activity, and not an artefact.

Before starting the actual adjustment of the trigger conditions, by performing the steps of FIG. 4, it may be useful to measure the Edi signal and the ventilator's breathing cycle for some breaths to compare the timing of the two. This comparison will indicate if the inspiration support is triggered too early or too late compared to the patient's own breathing activity, thereby indicating in which direction the trigger condition should be adjusted. Such a comparison can also be performed continuously, or at certain time intervals, to evaluate the need for adjusting the trigger conditions. If the timing of the Edi signal and the breathing support cycle differs less than a certain limit no adjustment is needed. If the difference in timing exceeds this limit an adjustment procedure as the one shown in FIG. 4 should be performed.

The difference between the inspired and expired volumes may be used to evaluate whether there is any leakage before the trigger condition is adjusted. If a considerable leak is present the sensitivity of the adjusted trigger should preferably be limited.

As mentioned above, the triggering may be based on pressure or flow of gas in the ventilator. In the case of a leakage, pressure triggering will be more suitable than flow triggering, since a leakage will cause a flow, even if there is no patient activity.

Although modifications and changes may be suggested by those skilled in the art, it is the intention of the inventors to embody within the patent warranted hereon all changes and modifications as reasonably and properly come within the scope of their contribution to the art.

We claim as our invention:

1-16. (canceled)

17. A computer-readable medium encoded with programming instructions, said medium being loaded into a computerized control unit of a ventilator that provides breathing support in a support mode to a patient, said programming instructions causing said computerized control unit to:
obtain a measurement value of a bioelectric signal representative of the breathing function of the patient;
determine, based on the bioelectric signal, at least one point in time at which the patient starts inhalation;
obtain a measurement value to be used for triggering an inspiration phase in the ventilator during said at least one point in time;
determine a trigger condition for the inspiration phase based on said measurement value; and
use said trigger condition to initiate inspiration when ventilating the patient in said support mode.

18. A computer-readable medium as claimed in claim 17 wherein said programming instructions cause said control unit to determine said trigger condition based on a plurality of measurement values, each obtained at a point-in-time when said patient begins inhalation for respectively different breaths.

19. A computer-readable medium as claimed in claim 17 wherein said programming instructions cause said control unit to adjust said trigger condition by an amount determined dependent on said measurement value.

20. A computer-readable medium as claimed in claim 17 wherein said programming instructions cause said control unit, after determining the start of inhalation and before obtaining said measurement value, to determine whether an inspiration phase was triggered in the ventilator before said start of inhalation and, if so, to delay said initiating of inspiration until a spontaneous attempt by the patient to breath is detected.

21. A computer-readable medium as claimed in claim 17 wherein said programming instructions cause said control unit to adjust said trigger condition incrementally to reduce a time difference between a start of an inspiration phase of said ventilator and the start of inspiration by the patient.

22. A computer-readable medium as claimed in claim 21 wherein said programming instructions cause said control unit to adjust said trigger condition in fixed increments.

23. A computer-readable medium as claimed in claim 21 wherein said programming instructions case said control unit to adjust said trigger condition in increments determined from a difference between said measurement value and said trigger condition.

24. A control unit for a ventilator operated by a computer-readable medium comprising programming instructions, said programming instructions configuring said control unit to:
obtain a measurement value of a bioelectric signal representative of the breathing function of the patient;
determine, based on the bioelectric signal, at least one point in time at which the patient starts inhalation;
obtain a measurement value to be used for triggering an inspiration phase in the ventilator during said at least one point in time;
determine a trigger condition for the inspiration phase based on said measurement value; and
use said trigger condition to initiate inspiration when ventilating the patient in said support mode.

25. A ventilator comprising:
a breathing circuit adapted for connection to a patient, said breathing circuit being operable in a support mode to assist breathing by the patient;
a control unit that operates said breathing circuit, said control unit being configured to obtain a measurement value of a bioelectric signal representative of the breathing function of the patient, determine, based on the bioelectric signal, at least one point in time at which the patient starts inhalation, obtain a measurement value to be used for triggering an inspiration phase in the ventilator during said at least one point in time, determine a trigger condition for the inspiration phase based on said measurement value, and use said trigger condition to initiate inspiration when ventilating the patient in said support mode.

26. A method for operating a computerized control unit of a ventilator that provides breathing support in a support mode to a patient, said method comprising the steps of:
obtaining a measurement value of a bioelectric signal representative of the breathing function of the patient;
in a processor, determining, based on the bioelectric signal, at least one point in time at which the patient starts inhalation;
obtaining a measurement value to be used for triggering an inspiration phase in the ventilator during said at least one point in time;
in said processor, determining a trigger condition for the inspiration phase based on said measurement value; and
from said processor, using said trigger condition to initiate inspiration when ventilating the patient in said support mode.

27. A method as claimed in claim 26 comprising determining said trigger condition based on a plurality of measurement values, each obtained at a point-in-time when said patient begins inhalation for respectively different breaths.

28. A method as claimed in claim 26 comprising, in said processor, adjusting said trigger condition by an amount determined dependent on said measurement value.

29. A method as claimed in claim 26 comprising, after determining the start of inhalation and before obtaining said measurement value, determining whether an inspiration phase was triggered in the ventilator before said start of inhalation and, if so, delaying said initiating of inspiration until a spontaneous attempt by the patient to breath is detected.

30. A method as claimed in claim 26 comprising, in said processor, adjusting said trigger condition incrementally to reduce a time difference between a start of an inspiration phase of said ventilator and the start of inspiration by the patient.

31. A method as claimed in claim 30 comprising adjusting said trigger condition in fixed increments.

32. A method as claimed in claim 30 comprising adjusting said trigger condition in increments determined from a difference between said measurement value and said trigger condition.
